(19) United States
(12) Patent Application Publication
(10) Pub. No.: US 2025/0213129 A1
(43) Pub. Date: Jul. 3, 2025

(54) MONITORING A CONDITION OF A LIVING ORGANISM

(71) Applicant: TRINAMIX GMBH, Ludwigshafen Am Rhein (DE)

(72) Inventors: Muhammad Muneeb HASSAN, Ludwigshafen am Rhein (DE); Christian LENNARTZ, Ludwigshafen am Rhein (DE)

(21) Appl. No.: 18/850,846

(22) PCT Filed: Apr. 21, 2023
(86) PCT No.: PCT/EP2023/060390

§ 371 Date: Sep. 25, 2024

(30) Foreign Application Priority Data
Apr. 26, 2022 (EP) 22169932.5

(51) Int. Cl. A61B 5/026; A61B 5/00; G06T 7/00

(52) U.S. Cl. CPC A61B 5/0261; A61B 5/7267; G06T 7/0016; G06T 2207/20081; G06T 2207/30104

(57) ABSTRACT

(a) receiving an image set including at least two reflection images generated at different points in time and an indication of at least one interval between at least two different points in time, where a reflection image is generated while the living organism is illuminated by patterned coherent electromagnetic radiation and includes a pattern with at least one pattern feature, and, where the reflection image shows at least one pattern feature formed by illuminating at least a part of the living organism by the patterned coherent electromagnetic radiation;

(b) determining a feature contrast of the at least two pattern features;

(c) determining a condition measure of the living organism based on the feature contrast and the indication of the at least one interval; and

(d) outputting the condition measure.

### DESCRIPTION

The present invention is in the field of monitoring a condition of a living organism. In particular, it relates to a method for monitoring a condition of a living organism, non-transitory computer-read-able data medium storing a computer program including instructions for executing steps of the method, a system for monitoring a condition of a living organism, use of a signal indicating a condition-based action controlling a condition monitoring system and use of a condition measure for operating a condition monitoring system.

### BACKGROUND

Techniques for remote monitoring of a heart rate, stress or blood pressure indicating a condition of a living organism are generally known such as photoplethysmography (PPG) and ballistocardiography (BCG).

PPG can be used to detect blood volume changes. Usually changes in intensity due to light absorption are observed using visible light. These changes can allow deriving information about the cardiovascular system. PPG can be used in a wide range of medical applications such as for monitoring heart rate and cardiac cycle, monitoring respiration, monitoring depth of anesthesia, monitoring hypo-and hypervolemia, monitoring blood pressure, see e.g. en.wikipedia.org/wiki/Photoplethysmogram. Ballistocardiography (BCG) is a non-invasive method which is based on measurement of body motion generated by the ejection of the blood at each cardiac cycle, see L. Giovangrandi et al. “Ballistocardiography—A Method Worth Revisiting” Conf Proc IEEE Eng Med Biol Soc. 2011; 2011: 4279-4282, doi: 10.1109/IEMBS.2011.6091062.

However, these techniques usually require staying near to the living organism due to usage of visible light. Further uniform lighting over the region is required. This may result in severe drawbacks when imaging non-skin areas e.g. background due to movements, and high costs.

It was hence the object of the present invention to overcome these shortcomings. In particular, a method for monitoring a condition of a living organism was aimed which provides full mobility to the living organism without any distraction. This method should be fast, require inexpensive hardware and should not involve physical contact with the living organism.

### SUMMARY

These objects were achieved by the present invention. In one aspect it relates to a method for monitoring a condition of a living organism comprising:
(a) receiving an image set comprising at least two reflection images generated at different points in time and an indication of at least one interval between at least two different points in time, wherein a reflection image shows at least a part of the living organism while the living organism is illuminated by coherent electromagnetic radiation and comprises a pattern with at least one pattern feature, and,
(b) determining a feature contrast of the at least two pattern features,
(c) determining a condition measure of the living organism based on the feature contrast and the indication of the at least one interval,
(d) outputting the condition measure.

In another aspect, it relates to a method for monitoring a condition of a living organism comprising:
(a) receiving an image set comprising at least two reflection images generated at different points in time and an indication of at least one interval between at least two different points in time, wherein a reflection image is generated while the living organism is illuminated by patterned coherent electromagnetic radiation, and, wherein the reflection image shows at least one pattern feature formed by illuminating at least a part of the living organism by the patterned coherent electromagnetic radiation, and,
(b) determining a feature contrast of the at least two pattern features,
(c) determining a condition measure of the living organism based on the feature contrast and the indication of the at least one interval,
(d) outputting the condition measure.

The invention is based on the recognition that a moving body fluid or moving particles in the body of a living subject, such as blood cells, in particular, red blood cells, interstitial fluid, transcellular fluid, lymph, ions, proteins and nutrients, may cause a motion blur to reflected light while the remaining parts of the body are still and hence do not cause motion blur. Thus, if coherent electromagnetic radiation is reflected by moving scattering particles like red blood cells, the pattern fluctuates and the pattern features become blurred. As a consequence of this blurring, the features contrast decreases. Therefore, the pattern and the feature contrast derived from the pattern contain information about whether or not the illuminated object is in motion. Feature contrast may comprise at least one feature contrast value. Feature contrast values are generally distributed between 0 and 1. Determining a feature contrast may include determining a feature contrast value. In particular, determining a feature contrast of the at least two pattern features may refer to determining a first feature contrast in relation to the first pattern feature of the at least two pattern features and determining a second feature contrast in relation to the second pattern feature of the at least two pattern features. Preferably, determining a feature contrast of the at least two pattern features may refer to determining a first feature contrast value in relation to the first pattern feature of the at least two pattern features and determining a second feature contrast value in relation to the second pattern feature of the at least two pattern features.

When illuminating an object, the value 1 may represent no motion and the value 0 may represent the fastest motion of particles thus causing the most prominent blurring of the pattern features. Coherent electromagnetic radiation refers to electromagnetic radiation that is able to exhibit interference effects. It may also include partial coherence, i.e. a non-perfect correlation between phase values. Preferably, the coherent electromagnetic radiation may be in the infrared range, most preferably in the near-infrared range. In particular, the coherent electromagnetic radiation is patterned coherent electromagnetic radiation. Illuminating at least a part of the living organism with patterned coherent electromagnetic radiation may result in projecting at least one pattern feature onto the living organism. A reflection image generated while illuminating the living organism with patterned coherent electromagnetic radiation may show the at least one pattern feature. Illuminating at least a part of the living organism with patterned coherent electromagnetic radiation may allow an interaction of the patterned coherent electromagnetic radiation with the living organism, in particular with the skin of the living organism. Interaction of the patterned coherent electromagnetic radiation with the living organism, in particular with the skin of the living organism may result in the formation of speckles. Speckle may be a contrast variation, in particular a local contrast variation of the patterned coherent electromagnetic radiation, in particular of the area illuminated by the patterned coherent electromagnetic radiation. Followingly, the pattern feature may comprise a plurality of speckles and/or contrast variations. Determining the feature contrast may refer to determining the contrast variations with respect to the pattern features associated with the different points in time. Determining the contrast variations may refer to determining at least two feature contrast values, in particular a first feature contrast value associated with the first reflection image and a second feature contrast value associated with the second reflection image.

The present invention provides means for providing efficient and robust way for monitoring a condition of a living organism. The feature contrast of a plurality of reflection images can be used to determine a condition measure such as the heartbeat, blood pressure, aspiration level or the like. E.g. the heartbeat is a sensitive measure for the condition of a living organism, especially the stress level or similar factors indicated by the condition measure. Thus, monitoring the living organism allows for identifying situations where the living organism is e.g. stressed and corresponding actions may be taken on the basis of the condition measure determined. Identifying these situations is especially important where critical condition measures provide a health or security risk. Examples for these situations may be a driver controlling a vehicle, a user using a virtual reality headset or a person having to take a far-reaching decision. By identifying such situations, the security risk and the health risk in these situations is decreased.

Furthermore, the invention presented herein makes use of inexpensive hardware for monitoring the living organism. In addition to that, the invention is easy to integrate and conduct and no direct contact to a living organism is required by providing at the same time reliable results. Thus, the living organism is not limited in any way by the monitoring and by deploying light in the IR range the living organism may not recognize the monitoring being performed. Therefore, the living organism is not distracted by light or feels watched by deploying the methods, systems, computer-readable storage media and use of signals disclosed herein.

A living organism is an individual form of life, preferably humans or animals. The living organism may be characterized by body fluids flowing through the body of the living organism.

In another aspect, the invention relates to a non-transitory computer-readable data medium storing a computer program including instructions for executing steps of the method according to any one of the preceding claims.

In another aspect, the invention relates to a system for monitoring a condition of a living organism comprising:
(a) an input for receiving an image set comprising at least two reflection images generated at different points in time and an indication of at least one interval between at least two different points in time, wherein a reflection image shows at least a part of the living organism while the living organism is illuminated by coherent electromagnetic radiation and comprises a pattern with at least one pattern feature, and,
(b) a processor for
   determining a feature contrast of the at least two pattern features and
   determining a condition measure of the living organism based on the feature contrast and the indication of the at least one interval,
(c) an output for outputting the condition measure.

In another aspect, the invention relates to use of a condition measure obtained by the method of any of the preceding method claims in a condition controlling system.

In another aspect, the invention relates to a condition controlling system comprising:
(a) an input for receiving a condition measure obtained by the method of any of the preceding method claims and a threshold associated with a critical condition measure,
(b) a processor for generating a signal indicating a condition-based action based on a comparison of the condition measure with the threshold associated with a critical condition measure,
(c) an output for outputting the signal indicating a condition-based action.

In another aspect, the invention relates to a method for monitoring a condition of a living organism comprising:
(a) receiving at least a first reflection image, a second reflection image and an indication of an interval between the time at which the first reflection image was generated and the time at which the second reflection image was generated, wherein the first reflection image and the second reflection image show at least a part of the living organism while it is illuminated by coherent electromagnetic radiation comprising a pattern with at least one pattern feature, and,
(b) determining a first feature contrast of the pattern feature in the first reflection image and a second feature contrast of the pattern feature in the second reflection image,
(c) determining a condition measure of the living organism based on the first feature contrast, the second feature contrast and the indication of the interval,
(a) outputting the condition measure.

In another aspect, the invention relates to a method for monitoring a condition of a living organism, the method comprising:
(a) receiving at least a first reflection image, a second reflection image and an indication of an interval between the time at which the first reflection image was generated and the time at which the second reflection image was generated, wherein the first reflection image and the second reflection image show at least a part of the living organism while it is illuminated by patterned coherent electromagnetic radiation, and, wherein the first reflection image and the second reflection image show at least one pattern feature formed by illuminating at least a part of the living organism by the patterned coherent electromagnetic radiation, and,
(b) determining a first feature contrast of the pattern feature in the first reflection image and a second feature contrast of the pattern feature in the second reflection image,
(c) determining a condition measure of the living organism based on the first feature contrast, the second feature contrast and the indication of the interval,
(d) outputting the condition measure.

In another aspect, the invention relates to a method for monitoring a condition of a living organism comprising:
(a) receiving at least a first reflection image, a second reflection image and an indication of an interval between the time at which the first reflection image was generated and the time at which the second reflection image was generated, wherein the first reflection image and the second reflection image are generated while at least a part of the living organism illuminated by patterned coherent electromagnetic radiation, and, wherein the first reflection image and the second reflection image shows at least one pattern feature formed by illuminating at least a part of the living organism by the patterned coherent electromagnetic radiation, and,
(b) determining a first feature contrast of the pattern feature in the first reflection image and a second feature contrast of the pattern feature in the second reflection image,
(c) determining a condition measure of the living organism based on the first feature contrast, the second feature contrast and the indication of the interval,
(d) outputting the condition measure.

Any disclosure and embodiments described herein relate to the methods, the systems, the uses, the computer program element lined out above and vice versa. Advantageously, the benefits provided by any of the embodiments and examples equally apply to all other embodiments and examples and vice versa.

The coherent electromagnetic radiation is patterned coherent electromagnetic radiation. Patterned coherent electromagnetic radiation may comprise a plurality of light beams, eg at least one light beam, preferably at least two light beams. A pattern feature may be associated with one light beam of the patterned coherent electromagnetic radiation. In particular, one pattern feature may be associated with one light beam of the patterned coherent electromagnetic radiation. Hence, projection of a light beam of the patterned coherent electromagnetic radiation onto a surface may result in a pattern feature. A pattern feature may refer to a light spot. In particular, a pattern feature may be caused by projecting coherent electromagnetic radiation onto the living organism.

A projection of patterned coherent electromagnetic radiation onto a regular surface may result in a pattern feature projected onto the regular surface independent of speckles. A projection of patterned coherent electromagnetic radiation onto a regular surface may result in a pattern feature projected onto the irregular surface comprising at least one speckle, preferably a plurality of speckles. Followingly, a pattern feature may comprise zero, one or more speckles depending on the surface the patterned coherent electromagnetic radiation is projected on. Skin may have an irregular surface. Hence the projection of patterned coherent electromagnetic radiation may result in the formation of speckles within the one or more pattern features. A pattern feature may be a result of the projection of a light beam associated with the patterned coherent electromagnetic radiation. A pattern feature refers to an arbitrarily shaped spot of coherent electromagnetic radiation. A pattern feature may refer to a contiguous area illuminated with coherent electromagnetic radiation. Projecting coherent electromagnetic radiation on an irregular surface results in the formation of speckles. Followingly, the pattern feature may comprise one or more speckles. A pattern feature may have add diameter between 0.5 mm and 5 cm, preferably 0.6 mm and 4 cm, more preferably, 0.7 mm and 3 cm, most preferably 0.4 and 2 cm.

For example patterned coherent electromagnetic radiation may be generated by a plurality of light emitters such as a VCSEL array. An emitter of the plurality of light emitters may emit one light beam. Hence, an emitter of the plurality of light emitters may be associated with the one pattern feature, with the formation of one pattern feature and/or with the projection of one pattern feature. Additionally or alternatively, patterned coherent electromagnetic radiation may be generated by one or more light emitters and an optical element such as a DOE or a metasurface element. The optical element may replicate the number of light beams associated with the one or more light emitters and/or may be suitable for replicating the number of light beams associated with the one or more light emitters. For example, the light emitter may be a laser.

A reflection image shows at least a part of the living organism while the living organism is illuminated by patterned coherent electromagnetic radiation. Thus, a reflection image shows a projection of patterned electromagnetic radiation onto a living organism. A projection of patterned coherent electromagnetic radiation may show at least one pattern feature, preferably at least two pattern features. Followingly, a reflection image shows at least one pattern feature formed by illuminating at least a part of the living organism by the patterned coherent electromagnetic radiation.

A pattern refers to a regular and/or irregular arrangement of pattern features. Followingly, a pattern may refer to a regular and/or irregular arrangement of one or more arbitrarily shaped spots of coherent electromagnetic radiation. A pattern may refer to at least one contiguous area illuminated with coherent electromagnetic radiation, preferably at least two, more preferably at least five, most preferably at least ten. A pattern may not refer to an arrangement of speckles. Further, the concept of pattern may be independent of the concept of contrast maps and/or the concept of paving the image for local contrast determination. A pattern may refer to a regular and/or irregular arrangement of one or more light beams. A pattern and or a pattern feature may be formed prior to the interaction of the patterned coherent electromagnetic radiation with the surface of the living Organism and/or independent of the interaction of the patterned coherent electromagnetic radiation with the surface of the living Organism.

In an embodiment, a reflection image may show at least two pattern features. Preferably, a reflection image may show five or more pattern features.

Patterned coherent electromagnetic radiation refers to coherent electromagnetic radiation with spatially varying intensity. In particular, patterned coherent electromagnetic radiation may form a pattern comprising at least one pattern feature while illuminating the living organism. Further, the intensity of the patterned coherent electromagnetic radiation may vary over time and/or the coherent electromagnetic radiation may be a continuous wave.

The usage of coherent electromagnetic radiation for evaluating the condition of a living Organism is advantageous because the living organism is illuminated with varying intensities, eg with light spots. In contrast to flood illumination, using patterned coherent electromagnetic radiation enables the sparing of light-sensitive regions such as the eyes. Furthermore, the feature contrast can be determined for specific pattern features and a mean value can improve the accuracy of the estimation of the condition of a living organism. Overall, the luminance of the pattern features can be increased better which results in an improved quality of the reflection images. Ultimately, the improved quality of the reflection images enables a more accurate estimation of the condition of a living organism.

The term “reflection image” as used herein is not limited to an actual visual representation of an object. Instead, a reflection image comprises data generated based on light reflected by an object being illuminated with light. Reflection image may comprise at least one pattern. Reflection image comprises at least one pattern feature. Reflection image may be comprised in a larger reflection image. A larger reflection image may be a reflection image comprising more pixels than the reflection image comprised in it. Dividing a reflection image into at least two parts may result in at least two reflection images. The at least two reflection images may comprise different data generated based on light reflected by an object being illuminated with light, e.g. one of the at least two reflection images may represent a living organisms nose and the other one of the at least two reflection images may represent a living organisms forehead. Reflection image may be suitable for determining a feature contrast for the at least one pattern feature. Reflection image may comprise a plurality of pixels. A plurality of pixels may comprise at least two pixels, preferably more than two pixels. For determining a feature contrast at least one pixel associated with the reflection feature and at least one pixel not associated with the reflection feature may be suitable, wherein reflection feature refers to pattern feature.

In particular, the term “reflection image” as used herein can refer to any data based on which an actual visual representation of the imaged object can be constructed. For instance, the data can correspond to an assignment of color or grayscale values to image positions, wherein each image position can correspond to a position in or on the imaged object. The reflection images or the data referred to herein can be two-dimensional, three-dimensional or four-dimensional, for instance, wherein a four-dimensional image is understood as a three-dimensional image evolving over time and, likewise, a two-dimensional image evolving over time might be regarded as a three-dimensional image. A reflection image can be considered a digital image if the data are digital data, wherein then the image positions may correspond to pixels or voxels of the image and/or image sensor. While generating the reflection image, the living organism may be illuminated with light, eventually being RGB light or preferably IR flood light and/or patterned light. Patterned light may comprise at least one pattern. Patterned light may be projected onto the living organism. Patterned light may comprise patterned coherent electromagnetic radiation.

In this context, “generating” also includes capturing and/or recording an image.

The term “pattern” as used herein refers, without limitation, to an arbitrary known or pre-determined arrangement comprising at least one arbitrarily shaped pattern feature. The pattern may comprise at least one pattern feature. The pattern may comprise an arrangement of periodic or non-periodic pattern features. The pattern can be at least one of the following: at least one quasi random pattern; at least one Sobol pattern; at least one quasiperiodic pattern; at least one point pattern, in particular a pseudo-random point pattern; at least one line pattern; at least one stripe pattern; at least one checkerboard pattern; at least one triangular pattern; at least one rectangular pattern; at least one hexagonal pattern or a pattern comprising further convex tilings. A pattern feature is at least a part of a pattern. Pattern feature may comprise at least partially an arbitrarily shaped symbol. The symbols can be any one of: at least one point; at least one line; at least two lines such as parallel or crossing lines; at least one point and one line; at least one arrangement of periodic pattern features; at least one arbitrary shaped pattern.

A feature contrast may represent a measure for a contrast of an intensity distribution within an area of a pattern, in particular within the area of a pattern feature. Additionally or alternatively, feature contrast may refer to a measure for a contrast associated with a pattern feature. The feature contrast may be determined of the at least two pattern features. The feature contrast may indicate at least two feature contrast values, wherein the first of the at least two feature contrast values may be associated with the first of the at least two reflection images and/or the first of the at least two pattern features and the second of the at least two feature contrast values may be associated with the second of the at least two reflection images and/or the second of the at least two pattern features. In particular, the first of the at least two pattern features, in particular the at least one first pattern feature may be associated with the first of the at least two reflection images, in particular the at least one first reflection image. In particular, the second of the at least two pattern features, in particular the at least one second pattern feature may be associated with the second of the at least two reflection images, in particular the at least one second reflection image. The feature contrast may be determined of the first pattern feature of the at least two reflection images and for the second pattern feature of the at least two reflection images. The feature contrast may be determined separately for the at least two pattern features of the at least two reflection images.

The feature contrast may indicate and/or may comprise determining at least two feature contrast values associated with the at least two pattern features. Determining the feature contrast of the at least two pattern features may include determining a first feature contrast value associated with the first pattern feature and determining a second feature contrast value associated with the second pattern feature. A feature contrast value may be determined by determining the ratio of a standard deviation of a pattern feature intensity and a mean of the pattern feature intensity. Pattern feature intensity may comprise intensity values associated with the corresponding pattern feature.

In particular, a feature contrast value K over an area of the pattern may be expressed as a ratio of standard deviation o to the mean pattern feature intensity <I>, i.e.,

$$K = \frac{σ}{< I >}$$

Feature contrast values are generally distributed between 0 and 1. Feature contrast may be determined based on at least one pattern feature. Followingly, at least two feature contrast values may be determined based on the at least two pattern features.

In some embodiments, for determining the feature contrast, the complete pattern of the reflection image may be used. Alternatively, for determining the feature contrast, a section of the pattern may be used. The section of the pattern, preferably, represents a smaller area of the pattern than an area of the pattern. The area may be of any shape. The section of the pattern may be obtained by cropping the reflection image.

The feature contrast may be different for different parts of a living organism. Different parts of the living organism may correspond to different parts of the reflection image. Followingly, the feature contrast may be different for different parts of a reflection image.

An image set may comprise a set of reflection images. Preferably, the image set comprises at least two reflection images. The set of images may be generated at different points in time.

In some embodiments, at least one or more than one feature contrast values may be determined of the at least one pattern feature. A feature contrast value may correspond to a numerical value of a feature contrast.

A condition measure is a measure suitable for determining the condition of a living organism. A condition of a living organism may be a physical and/or mental condition. A physical condition may be associated with physical stress level, fatigue, excitation, suitability of performing a certain task of a living organism or the like. A mental condition may be associated with mental stress level, attentiveness, concentration level, excitation, suitability of performing a certain task of a living organism or the like. Such a certain task may require concentration, attention, wakefulness, calming or similar characteristics of the living organism. Examples for such a task can be controlling machinery, vehicle, mobile device or the like, operating on another living species, activities relating to sports, playing games, tasks in an emergency case, making decisions or the like. Condition measures indicate a condition of a living organism. Condition measures may be one or several of the following: heart rate, blood pressure, aspiration level or the like. In some embodiments, the condition of a living organism may be critical corresponding to a high value of the condition measure and the condition of a living organism may be non-critical corresponding to a low value of the condition measure. Followingly, the critical condition measure according to these embodiments may be equal or lower than a threshold and a non-critical condition measure may be lower than a threshold. In other embodiments, the condition of a living organism may be critical corresponding to a low value of the condition measure and the condition of a living organism may be non-critical corresponding to a high value of the condition measure. Followingly the critical condition measure according to these embodiments may be equal or higher than a threshold and a non-critical condition measure may be lower than a threshold. A critical condition measure may be associated with a high stress level, low attentiveness, low concentration level, high fatigue, high excitation, low suitability of performing a certain task of the living organism or the like. A non-critical condition measure may be associated with a low stress level, high attentiveness, high concentration level, low fatigue, low excitation, high suitability of performing a certain task of the living organism or the like.

The condition measure of a living organism may be determined based on the motion of a body fluid, preferably blood, most preferably red blood cells. The motion of body fluids is not constant over time but changes due to activity of parts of the living organism, e.g the heart. Such a change in motion may be determined based on a change in feature contrast over time. A high difference between values of feature contrast at different points in time may be associated with a fast change in motion. A low difference between values of feature contrast at different points in time may be associated with a slow change in motion. The change in motion of a body fluid, preferably blood, may be periodically associated with a corresponding motion frequency. Accordingly, the feature contrast may change periodically with the corresponding motion frequency. The motion frequency may correspond to the length of a period associated with the periodic change in feature contrast. In some embodiments, half of a period may be comprised in the at least two reflection images. In other embodiments, one or several periods may be comprised in the at least two reflection images. Preferably, pattern feature associated with the same part of a living organism may be used for determining the condition of a living organism. This is advantageous due to the fact that the blood perfusion and thus, the feature contrast across different parts of the body varies. In some embodiments, at least one condition measure may be determined based on the feature contrast.

The image set may comprise a time series. The time series may comprise reflection images separated by a constant time interval associated with an imaging frequency or changing time intervals. Preferably, the time series is constituted such that the imaging frequency is at least twice the motion frequency. This is known as the Nyquist theorem. For higher resolution more reflection images than at least required by the Nyquist theorem may be received.

For the determination of the condition measure, an indication of an interval between the different points in time where the at least two reflection images are generated is received. The indication of the interval comprises measure(s) suitable for determining the time between the different points in time where the at least two reflection images are generated.

A frequency is a reciprocal value of the length of a period. The length of a period may be determined by the interval between two reflection images comprising a share of the period of the heart beating or the heart cycle. In a normal human at rest the heart beats between 60 and 80 times per minute corresponding to a resting heart rate of 60 to 80 beats per minute (bpm). The resting heart rate may be lower, e.g. if the human is sportive or suffers from bradycardia. In situation where the human is active, the heart rate may increase up to 230 bpm. Animals may have heart rates ranging from 6 to 1000 bpm. The reflection images may be generated depending on the expected heart rate of the living organism examined. The interval between the reflection images may be chosen to be up to 10 seconds. In the case of a human, the interval may be chosen up to 2 seconds. Followingly, the imaging frequency may be chosen to be at least 12 reflection images per minute or at least 60 reflection images in the case of a human. In an example, the method may be used for determining the heart rate of a human. For this purpose, an imaging frequency of 60 images per minute may be chosen. As the feature contrast is determined, one may recognize that the imaging frequency may be too low. In such a case, the imaging frequency may be increased such that the condition measure may be determined. Alternatively, the imaging frequency for imaging a human may be chosen to be a high frequency such as 460 images per minute. A heart rate may be determined based on the at least two reflection images and an indication of the interval between the at least two different points in time indicating an interval of 0.13 seconds. In the example, the human may have a heart rate in the range of 60 to 80 bpm. Followingly, the imaging frequency may be adjusted according to expected and/or predetermined condition measures.

In an example, the interval may comprise a half, a full, double length of a period or the like. The interval may be between at least two reflection images. Followingly, the at least two reflection images may be separated by a half, a full, double length of a period or the like. In the exemplary case of three reflection images indication of one or two different intervals may be received. If more than two reflection images are received, the indication of the interval may comprise an indication of an interval between the first and the second reflection image and/or an interval between the first and the third reflection image (or every other reflection image if more than three reflection images may be received) and/or an interval between the second and the third reflection image (or every other reflection image if more than three reflection images may be received). This applies accordingly to other scenarios with a different amount of reflection images as the skilled person will recognize. Measures for the indication of the interval may be at least two points in time corresponding to the different points in time where the at least two reflection images are generated and/or the time that passed between the different points in time and/or an imaging frequency associated with the generation of the reflection images. The at least two points in time may be determined based on a timestamp of the at least two reflection images. The imaging frequency may comprise a selected value. The imaging frequency may be selected based on the expected condition measure, e.g. an expected heart rate. Alternatively, the image frequency of a video may be used to determine an imaging frequency. An expected heart rate may comprise a heart rate associated with the living organism monitored. In some embodiments, estimation of condition measure may be used to select the imaging frequency. An estimation of condition measure may take the living species and its surrounding into account.

In an embodiment, the image data set may comprise at least one first reflection image and at least one second reflection image. The at least one first reflection image and the at least one second reflection image may be generated at different points in time, in particular at at least two different points in time. The at least one first reflection image and the at least one second reflection image may be generated while the living organism is illuminated by patterned coherent electromagnetic radiation. The at least one first reflection image may show at least one first pattern feature, preferably at least two first pattern features, formed by illuminating at least a part of the living organism by the patterned coherent electromagnetic radiation. The at least one second reflection image may show at least one second pattern feature, preferably at least two second pattern features, formed by illuminating at least a part of the living organism by the patterned coherent electromagnetic radiation. In particular, the at least one first pattern feature and the at least one second pattern feature may be associated with the same body part of the living organism.

A feature contrast may be determined of the at least one first pattern feature and the at least one second pattern feature. The feature contrast may indicate a first feature contrast value associated with the at least one first pattern feature and a second feature contrast value associated with the at least one second pattern feature. A condition measure may be determined based on the feature contrast and the indication of the at least one interval by providing the feature contrast of the at least one first pattern feature and the at least one second pattern feature and the indication of the at least one interval between the generation of the at least one first reflection image and the at least one second reflection image to a data-driven model, wherein the data-driven model is parametrized on a training data set including historical feature contrasts indicating a plurality of first feature contrast values associated with a plurality of first pattern features and a plurality of second feature contrast values associated with the a plurality of second pattern features, a plurality of historical indications of the at least one interval and a plurality of historical condition measures.

In an embodiment, the condition measure of the living organism may be determined based on the feature contrast and the indication of the at least one interval by providing the feature contrast of the at least two pattern features and the indication of the at least one interval between the generation of the at least two pattern images to a data-driven model, wherein the data-driven model is parametrized on a training data set including historical feature contrasts, historical indications of the at least one interval and historical condition measures. The feature contrast may indicate at least two feature contrast values associated with the at least two pattern features.

In an embodiment, a condition measure of the living organism based on the first feature contrast, the second feature contrast and the indication of the interval may be determined by providing the first feature contrast, the second feature contrast and the indication of the interval to a data-driven model, wherein the data-driven model may be parametrized based on a training data set including historical first feature contrasts, historical second feature contrasts, historical indications of the at least one interval and historical condition measures.

Providing the feature contrast may include providing a first feature contrast value and a second feature contrast value. The data-driven model may be parametrized based on the training data set to provide and/or output a condition measure based on being provided with the feature contrast of the at least two pattern features and the indication of the at least one interval between the generation of the at least two pattern images. The data-driven model may be trained based on the training data set including historical feature contrasts, historical indications of the at least one interval and historical condition measures to provide and/or output a condition measure based on being provided with the feature contrast of the at least two pattern features and the indication of the at least one interval between the generation of the at least two pattern images. The condition measure of the living organism may be determined based on the feature contrast and the indication of the at least one interval by providing the feature contrast of the at least two pattern features and the indication of the at least one interval between the generation of the at least two pattern images to a data-driven model, wherein the data-driven model is trained on a training data set including historical feature contrasts, historical indications of the at least one interval and historical condition measures. The data-driven model may receive the feature contrast and the indication of the at least one interval at an input layer and/or may provide a condition measure based on having received the feature contrast and the indication of the at least one interval at an input layer. The data-driven model may comprise at least one machine learning architecture, in particular a deep learning architecture. For example, the data-driven model may be a neural network such as a CNN, in particular a 3D CNN, or a transformer. Further, the data-driven model may be a transformer network.

In an embodiment, the at least two pattern features may be associated with the at least two reflection images. Preferably, a first pattern feature of the at least two pattern features may be shown in the first reflection image of the at least two reflection images and a second pattern feature of the at least two pattern features may be shown in the second reflection image of the at least two reflection images. A feature contrast may be determined of the at least two pattern features including the first pattern feature and the second pattern feature. In particular, determining a feature contrast of the at least two pattern features including the first pattern feature and the second pattern feature may include determining a first feature contrast including a first feature contrast value of the first pattern feature of the first reflection image of the at least two reflection images and a second feature contrast value of the second pattern feature of second first reflection image of the at least two reflection images. Determining a condition measure of the living organism based on the feature contrast and the indication of the at least one interval may include determining a condition measure based on a first feature contrast value of the first pattern feature of the first reflection image of the at least two reflection images and a second feature contrast value of the second pattern feature of second first reflection image of the at least two reflection images.

In some embodiments, the condition measure is determined using an algorithm that may implement a mechanistic model or a data-driven model. The mechanistic model, preferably, reflects physical phenomena in mathematical form, e.g., including first-principle models. A mechanistic model may comprise a set of differential equations that describe an interaction between the object and the coherent electromagnetic radiation thereby resulting in a specific condition measure. In particular, flow of a fluid and/or the geometry of the object may be represented by the mechanistic model. The mechanistic model may comprise relations between the at least two reflection images, the indication of the point in time and the condition measure. For this purpose, the relations may be suitable for determining the time interval between the at least two reflection images and determining a motion frequency corresponding to the beats per time unit (heart rate). Based on the required input to the mechanistic model leading to a feature contrast as determined from the pattern of the at least two reflection images, an associated condition measure can be determined with the mechanistic model. Including a pulse wave analysis into the mechanistic model may be suitable for determining the blood pressure as another condition measure. To do so, the velocity of the pulse wave may be determined. This information may be comprised in the at least two reflection images and the indication about an interval. The absorption and reflection behaviour of the part of the living organism may indicate the aspiration level comprised in the at least one pattern feature of the reflection images. Oxygen-rich blood absorbs and thus, reflects light differently than oxygen-poor blood. Other condition measures may be determined by deploying relations between pattern features and the condition measure.

Preferably, the data-driven model is a parametrized classification model. The classification model may comprise at least one machine-learning architecture and model parameters. For example, the machine-learning architecture may be or may comprise one or more of: linear regression, logistic regression, random forest, piecewise linear, nonlinear classifiers, support vector machines, naive Bayes classifications, nearest neighbours, neural networks, convolutional neural networks, generative adversarial networks, support vector machines, or gradient boosting algorithms or the like. In the case of a neural network, the model can be a multi-scale neural network or a recurrent neural network (RNN) such as, but not limited to, a gated recurrent unit (GRU) recurrent neural network or a long short-term memory (LSTM) recurrent neural network. The term “training”, also denoted learning, as used herein, is a broad term and is to be given its ordinary and customary meaning to a person of ordinary skill in the art and is not to be limited to a special or customized meaning. The term specifically may refer, with-out limitation, to a process of building the classification model, in particular determining and/or updating parameters of the classification model. The classification model may be at least partially data-driven. For example, the classification model may be based on experimental data, such as data determined by illuminating a plurality of living organisms such as humans and recording the reflection images. For example, the training may comprise using at least one training dataset, wherein the training data set comprises reflection images, e.g. of a plurality of humans with known condition measures. For example, if the neural network is a feedforward neural network such as a CNN, a backpropagation-algorithm may be applied for training the neural network. In case of a RNN, a gradient descent algorithm or a backpropagation-through-time algorithm may be employed for training purposes.

In another aspect the invention relates to a non-transitory computer-readable data medium storing a computer program including instructions for executing steps of the method according to any one of the preceding claims.

Any disclosure and embodiments described herein relate to the methods, the systems, the computer readable media lined out above and vice versa. Advantageously, the benefits provided by any of the embodiments and examples equally apply to all other embodiments and examples and vice versa. The system may be suitable for carrying out the steps of the method.

“Computer-readable data medium” refers to any suitable data storage device or computer readable memory on which is stored one or more sets of instructions (for example software) embodying any one or more of the methodologies or functions described herein. The instructions may also reside, completely or at least partially, within the main memory and/or within the processor during execution thereof by the computer, main memory, and processing device, which may constitute computer-readable storage media. The instructions may further be transmitted or received over a network via a network interface device. Computer-readable data medium include hard drives, for example on a server, USB storage device, CD, DVD or Blue-ray discs. The computer program may contain all functionalities and data required for execution of the method according to the present invention or it may provide interfaces to have parts of the method processed on remote systems, for example on a cloud system. The term “non-transitory” has the meaning that the purpose of the data storage medium is to store the computer program permanently, in particular without requiring permanent power supply.

An input comprises of one or more of serial or parallel interfaces or ports, USB, Centronics Port, FireWire, HDMI, Ethernet, Bluetooth, RFID, Wi-Fi, USART, or SPI, or analogue interfaces or ports such as one or more of ADCs or DACs, or standardized interfaces or ports to further devices.

A “processor” is a local processor comprising a central processing unit (CPU) and/or a graphics processing units (GPU) and/or an application specific integrated circuit (ASIC) and/or a tensor processing unit (TPU) and/or a field-programmable gate array (FPGA). The processor may also be an interface to a remote computer system such as a cloud service. The processor may include or may be a secure enclave processor (SEP). An SEP may be a secure circuit configured for processing the reflection images. A “secure circuit” is a circuit that protects an isolated, internal resource from being directly accessed by an external circuit. The processor may be an image signal processor (ISP) and may include circuitry suitable for processing reflection images, in particular, reflection images received by the input or generated with a camera. In some embodiments, the processor may be configured to receive reflection images from the camera.

An output is one or more of serial or parallel interfaces or ports, USB, Centronics Port, FireWire, HDMI, Ethernet, Bluetooth, RFID, Wi-Fi, USART, or SPI, or analogue interfaces or ports such as one or more of ADCs or DACs, or standardized interfaces or ports to further devices. In some embodiments, the output may be configured to output a signal indicating the condition-based action.

A system for monitoring a condition of a living organism comprises an input, a processor and an output. The system for monitoring a condition of a living organism may be suitable for carrying out the steps of the methods described herein. The system may be or may be integrated into a monitoring device. Examples for monitoring devices can be mobile devices such as mobile phones like smartphones, smartwatches, tablet, laptops, headsets, especially virtual reality headsets, glasses, especially smart glasses. Monitoring devices can be integrated into machinery, vehicles, points of access, walls, furniture or the like.

A condition controlling system may be suitable for receiving a condition measure obtained by any of the preceding embodiments of the method described herein and a threshold. Such a condition controlling system can be operated without contact to the living organism. Hence, the condition of the living organism may be monitored unobservable to the living organism and without disturbance of the living organism. In addition, the hardware needed to implement a system or a method as described herein is low-cost standard hardware readily available without major adjustments. The condition controlling system comprises an input for receiving a condition measure and a threshold, a processor for generating a signal indicating a condition-based action based on a comparison of the condition measure with a threshold, an output for outputting the signal indicating a condition-based action. The condition controlling system may be suitable to initiate a condition-based action. The condition controlling system may be or may be integrated into a monitoring device. Examples for monitoring devices can be mobile devices such as mobile phones like smartphones, smartwatches, tablet, laptops, headsets, especially virtual reality headsets, glasses, especially smart glasses or any device suitable for carrying out the steps of the methods described herein. Monitoring devices can be integrated into machinery, vehicles, points of access, walls, furniture or the like. The connection may be established via a wired or wireless connection such as one of ethernet, USB, LAN, WLAN or the like. Examples for a controlling device may be a mobile device like smartphone, smartwatch, portable computer, tablet, board computer etc. A condition controlling system may be deployed in scenarios where a critical condition of a living organism can result in a security problem for the living organism and/or the surrounding. Examples for such scenarios can be at one of the following: a living organism controlling a device like a vehicle or machinery.

A condition-based action is a result of a comparison of the condition measure with the threshold. The condition measure may be determined as described herein. A signal indicating the condition-based action may be generated based on the comparison. The signal may be received by a condition controlling system. The condition-based action may be an advisory and/or interfering action. In some embodiments, the signal may indicate no action. If the condition measure of a living organism is below a threshold, the living organism should not be limited. The advisory action provides the living organism with advice. In some embodiments, the advisory action may provide another living organism with advice as the living organism from which the reflection images have been generated. In exemplary scenarios such as monitoring children, animals, humans with health issues, older humans, humans with a disability or the like humans responsible for taking care of the living organism may be notified. Humans responsible for taking care may be parents, caregiver, doctors, veterinarians or the like. The advisory action may comprise any form of providing advice in a form suitable for a living organism to recognize. Such advice may be for example advising the living organism to take a break, to drink and/or to eat, to change the conditions and/or the surrounding of the living organism, e.g. audio input, temperature, visual input, air circulation, etc. Examples for the form of advisory action can be visual through displaying functions, hearable through sound generating, e.g. a warning signal or tangible through vibrations. The interfering action may comprise any form of regulating the exterior. This is advantageous when the condition of a living organism is critical and can be improved by performing an interfering action. Such regulation may be for example, regulating the conditions and/or the surrounding of the living organism (e.g. audio input, temperature, visual input, air circulation, etc.), limiting the time during which the living organism may operate and/or control a mobile device and/or perform a certain task. In some scenarios, a preceding advisory action may be ignored by the living organism demanding interfering actions. In addition, very critical condition measures may be of higher risk and may be more adequately handled with interfering actions. Only slightly critical condition measures may be sufficiently handled with advisory action.

In one embodiment, a signal indicating a condition-based action based on a comparison of the condition measure with a threshold may be generated and the signal may be output.

A threshold is a predetermined value indicating the lowest value of a condition measure being critical. In some embodiments, the threshold may be stored locally in a memory. In other embodiments, the threshold may be provided from an external source such as a cloud server. The threshold may be received by the input. The threshold can be selected based on the required certainty that a living organism's condition of a living organism is critical, so minimizing the false positive rate. This comes at the cost of identifying too many situations as critical, i.e. yield a high false negative rate. The threshold is hence usually a compromise between minimizing the false positives rate and keeping the false negative rate at a moderate level. The threshold may be selected to obtain an equal or close to equal false negative rate and false negative rate. The threshold may be suitable for a comparison with the condition measure. The threshold may be a numerical value. In some embodiments, the threshold may be a value on a scale. Such a scale can range between two arbitrary values, preferably 0 and an arbitrary value, most preferably between 0 and 1. In other embodiments, the threshold may be a predetermined value of a condition measure. The threshold may be associated with a critical condition measure. The threshold may comprise a critical condition measure.

In one embodiment, the threshold may be specific for the living organism. The threshold can be a predetermined value indicating the lowest value of a condition measure necessary for triggering a condition-based action. To ensure safety, a correct definition of the threshold is necessary. Depending on factors such as age, sex, weight, size, genetics and so on heart rate, blood pressure, aspiration level or the like may be different than the median values. Thus, the threshold may be adjusted to the factors specific for the living organism. The threshold may be selected from predetermined values depending on the factors. In some embodiments, the threshold may be determined based on data generated during the monitoring of the living organism. For these purposes, algorithms such as data-driven or mechanistic algorithms may be deployed.

In another embodiment, the at least two reflection images may be generated. The at least two reflection images may be received from a camera. In particular, the image data set comprising the at least two reflection images may be received from a camera. The image data set comprising the at least two reflection images may be generated with a camera. The term “camera” specifically may refer, without limitation, to a device having at least one imaging element configured for recording or recording spatially resolved one-dimensional, two-dimensional or even three-dimensional optical data or information. The camera may be a digital camera. As an example, the camera may comprise at least one camera chip, such as at least one CCD chip and/or at least one CMOS chip configured for recording images. The camera may be or may comprise at least one near infrared camera and/or an RGB camera. Furthermore, the camera, besides the at least one camera chip or imaging chip, may comprise further elements, such as one or more optical elements, e.g. one or more lenses.

In another embodiment, the living organism may be illuminated with coherent electromagnetic radiation comprising a pattern with at least one pattern feature. The living organism may be illuminated with an illumination source. The illumination can be achieved by using a projector or illumination source which emits the light pattern onto the part of the living organism. The illumination source may comprise at least one light source. The illumination source may comprise a plurality of light sources. The illumination source is suitable for illuminating an object. The illumination source may comprise an artificial illumination source, in particular at least one laser source and/or at least one incandescent lamp and/or at least one semiconductor light source, for example, at least one light-emitting diode, in particular an organic and/or inorganic light-emitting diode. As an example, the light emitted by the illumination source may have a wavelength of 300 to 1100 nm, especially 500 to 1100 nm. Additionally or alternatively, light in the infrared spectral range may be used, such as in the range of 780 nm to 3.0 μm. Specifically, the light in the part of the near infrared region where silicon photodiodes are applicable specifically in the range of 700 nm to 1100 nm may be used. Using light in the near infrared region allows that light is not or only weakly detected by human eyes and is still detectable by silicon sensors, in particular standard silicon sensors. The illumination source may be adapted to emit light at a single wavelength. In other embodiments, the illumination may be adapted to emit light with a plurality of wavelengths allowing additional measurements in other wavelengths channels. The light source may be or may comprise at least one multiple beam light source. For example, the light source may comprise at least one laser source and one or more diffractive optical elements (DOEs). The illumination source may comprise at least one line laser. The line laser may be adapted to send a laser line to the object, for example a horizontal or vertical laser line. The illumination source may comprise a plurality of line lasers. For example, the illumination source may comprise at least two line lasers which may be arranged such that the illumination pattern comprises at least two parallel or crossing lines. The illumination source may comprise the at least one light projector adapted to generate a cloud of points such that the illumination pattern may comprise a plurality of point pattern. The illumination source may comprise at least one mask adapted to generate the illumination pattern from at least one light beam generated by the illumination source.

In an embodiment, the reflection image may be generated with a camera. Preferably, the camera may comprise a polarizer. The polarizer may be suitable for selecting the polarization of the reflection of the patterned coherent electromagnetic radiation from the living organism. Depending on the penetration depth, the patterned coherent electromagnetic radiation may be polarized differently. Using a polarizer eliminates the light directly reflected from the surface being disturbing for the analysis of blood flow. Followingly, the analysis is more robust when using a polarizer while obtaining the reflection images.

In one embodiment, the reflection images may be separated by a constant time interval associated with an imaging frequency or changing time intervals.

In another embodiment, the imaging frequency may be at least twice the motion frequency being associated with the expected periodic motion of a body fluid. An expected periodic motion of a body fluid may be associated with an expected periodic motion of the body fluid. The motion of the body fluid may be estimated based on the living organism and its surrounding.

In another embodiment, the data-driven model may be used to determine a condition measure of the living organism based on the feature contrast.

In some embodiments, the living organism may be in motion relative to the camera, wherein the motion may not correspond to the motion of blood. In such a case, the image may be motion corrected based on motion tracking data associated with the movement of the living organism. The motion tracking data may be received. The motion tracking data may comprise at least two contour images generated while the at least two reflection images may be generated and/or video while the at least two reflection images may be generated. A contour image may refer to an image showing the contour of at least a part of the living organism. A contour image may be suitable for identifying at least a part of the living organism. For example, a contour image of at least a part of the living organism may show the shape of a body part such as a hand, a face or the like. Additionally or alternatively, the motion tracking data may include an indication of an area in relation to at least a part of the living organism being illuminated with patterned coherent electromagnetic radiation, an indication of a shift of an area in relation to at least a part of the living organism being illuminated with patterned coherent electromagnetic radiation between the at least two different points in time. The motion tracking data may be used to identify a movement associated with the living organism while the at least two reflection images may be generated. A measure for the velocity of the movement of the living organism may be determined based on the indication of the at least one interval between the generation of the at least two reflection images and a distance estimate in relation to the motion tracking data. For example, the distance between two eyes in an image may be estimated. This estimation can be used to derive a distance associated with the movement of the living organism. A correction factor may be determined based on the measure for the velocity of the movement of the living organism. The correction factor may be a number indicating the contribution of the movement of the living organism to the feature contrast of the at least two reflection images. By correcting the feature contrast according to the correction factor a corrected feature contrast may be determined. The condition measure may be determined based on the corrected feature contrast. Correcting the feature contrast according to the correction factor a corrected feature contrast may include linking the correction factor and the feature contrast by a mathematical operation such as multiplication, division, summing, dividing or the like. Further details on motion correction and/or motion tracking can be found in US2022039679A1.

Such a correction aims at correcting the reflection image for the motion not related to blood perfusion. This can be done by tracking the motion of the living organism. By tracking the motion, a compensation factor can be determined suitable for subtracting from the feature contrast of a reflection image. The larger the movement of the living organism that is not related to blood perfusion, the larger the correction factor for subtracting is. The correction factor may be different for different parts of the reflection image. By doing so, the correction factor accounts for inhomogeneous movements over the reflection image. This is advantageous since it enables the use of reflection images where the living organism was in motion and thus, the reflection images do not need to be discarded and generating more reflection images can be avoided. Followingly, less data needs to be generated, processed and eventually stored, ultimately saving energy consumption.

In an embodiment, the patterned coherent electromagnetic radiation may be associated with a wavelength between 900 nm and 1000 nm and/or between 1100 nm and 1200 nm. Preferably, the patterned coherent electromagnetic radiation may have a wavelength between 920 nm and 980 nm and/or between 1110 nm and 1190 nm. More preferably, the patterned coherent electromagnetic radiation may have a wavelength between 930 nm and 950 nm and/or between 1120 nm and 1180 nm. Within this range, the sunlight has a band gap and the light is invisible to a human. Followingly, humans are not distracted when using the selected wavelength range and methods and systems are insensitive towards sun light. This enables the application of the invention as described herein with presence of ambient light und outside of closed rooms. Hence, the invention provides a mobile and versatile measurement of a condition of a living organism.

In an embodiment, the patterned coherent electromagnetic radiation may be emitted from an illumination source. Further, the reflection images may be generated with a camera. The distance between the living organism and the camera and/or the distance between the living organism and the illumination source may be between 5 cm and 150 cm. Preferably, the distance between the living organism and the camera and/or the distance between the living organism and the illumination source may be between 15 cm and 100 cm. More preferably, the distance between the living organism and the camera and/or the distance between the living organism and the illumination source may be between 20 cm and 80 cm. Projecting light onto a living organism and/or generating an image of a living organism enables a contactless data generation for evaluating the condition of a living organism. Further, the above-identified distances enable the use of low-cost and readily available hardware, eg hardware that can easily be integrated into mobile devices such as a phone, laptop or watch. Thus, the invention provides a low-cost, mobile and widely applicable evaluation of a condition of a living organism.

In some embodiments, the at least one part of the living organism preferably shown in the at least two reflection images may correspond to the same part of the living organism. A part of a living organism may be suitable for reflecting at least one pattern feature. In particular, the part of the living organism may comprise at least a part of the skin of the living organism.

In some embodiments, the image set may comprise at least three reflection images and wherein the at least three reflection images may have been generated in a time series with a constant time interval associated with an imaging frequency or changing time intervals associated with a mean imaging frequency.

In some embodiments, the step of outputting the condition measure may be substituted by:
generating a signal indicating a condition-based action based on a comparison of the condition measure with a threshold associated with a critical condition measure,
outputting the signal indicating the condition-based action.

In particular, the threshold may be specific for the living organism. Depending on the individual form of life, the threshold may be different, similar to the condition measure being dependent on the individual form of life. The individual form of life may be for example human, dog, cat, elephant, pig, cow, fish, frog or the like. Due to their different anatomy, the condition measure such as the heart rate may be in different ranges. Usually, smaller animals like cats may have higher heart rates than larger living organisms such as human or whale. By regarding the individual form of life, the determination of the condition-based action may be improved. This provides a more accurate determination of a suitable condition-based action.

In some embodiments, the at least one part of the living organism may be shown in the at least two reflection images and may correspond to the same part of the living organism.

In some embodiments, the system may further comprise an illumination source.

In some embodiments, the system may further comprise a camera.

In an embodiment, the at least two reflection images may show the same part of the living Organism and or the at least two reflection images may be generated while the same part of the living Organism is illuminated with patterned coherent electromagnetic radiation.

In some embodiments, the signal indicating a condition-based action may be used in a condition controlling system.

Due to the benefits provided by this invention, the methods, systems, computer-readable storage media and use of signals can be applied in diverse fields. Exemplary fields for application can be automotive context, medical context, entertainment business, sports context or any other field where a critical condition of a living organism can result in a security problem for the living organism and/or the surrounding. In the automotive context, a driver may be the living organism. The driver can be monitored accurately and secure control over a vehicle can be ensured. Thereby, lowering the risk of accidents. In the medical context, the methods are especially well-suited for living organisms where a watch, clipper or any other device in direct contact with the living organism may be problematic. Examples are (newborn) babies, living organisms with large injuries or sleeping living organisms. Applying the invention in the entertainment business and the sports context can prevent an overstraining and/or overstressing of the living species. The living organism may be a human in this case. The methods, the signals, the condition measures and/or systems may be comprised and/or may be used in vr technology, especially in a vr headset.

Further possible implementations or alternative solutions of the invention also encompass combinations—that are not explicitly mentioned herein-of features described above or below in regard to the embodiments. The person skilled in the art may also add individual or isolated aspects and features to the most basic form of the invention.

It shall be understood that a preferred embodiment of the invention can also be any combination of the dependent claims or above embodiments with the respective independent claim. Further embodiments, features and advantages of the present invention will become apparent from the subsequent description and dependent claims, taken in conjunction with the accompanying drawings.

### BRIEF DESCRIPTION OF THE FIGURES

FIG. 1 illustrates a flow diagram of an example embodiment of a method for monitoring a living organism.

FIG. 2 illustrates a block diagram of an exemplary system for monitoring a living organism comprising an input, a processor and an output.

FIG. 3 shows a representation of the feature contrast as determined from reflection images at different points in time.

FIG. 4 illustrates a flow diagram of an example embodiment of a method for monitoring a living organism further comprising the generation of a signal indicating a condition-based action.

FIG. 5 illustrates an example implementation of embodiments of the invention in vehicles.

FIG. 6 illustrates an example embodiment of a reflection image (620, 640, 660, 680).

FIG. 7 illustrates an example embodiment of the methods and systems as described herein.

### DESCRIPTION OF EMBODIMENTS

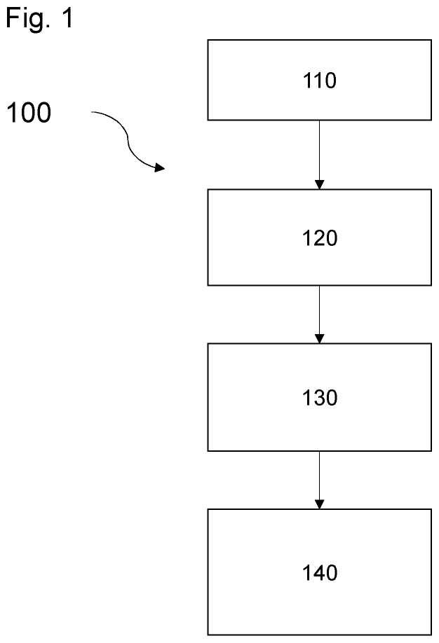

FIG. 1 illustrates a flow diagram of an example embodiment of a method for monitoring a living organism (100).

In a first step, at least two reflection images are received (110). The first reflection image may be generated at point t in time. The second reflection image may be generated at point t+p wherein p is the length of a period of the cardiac cycle associated with the heart rate. Furthermore, an indication of the interval between the different points in time where the reflection images may be generated are received (110). In the case of one reflection image at point t and another reflection image at point t+p the indication of the interval is suitable for determining the interval length of p. The feature contrast at point t and point t+p may be equal wherein the term equal is to be understood in the limitations of measurement uncertainty and/or biological variations. Condition measures underlie biological variations since the blood perfusion may deviate depending on various criteria. A data-driven model may be trained for compensating uncertainty and/or biological variations. A mechanistic model may be suitable for compensating uncertainty and/or biological variations. For a determination of the condition measure more than two reflection images may be received. In a time series, imaging the temporal evolution of the blood perfusion is periodic due to the periodic heartbeat. A third reflection image between two images separated temporally by one period length may be advantageously to ensure a change in motion during the length of one period. For example, the reflection images may be generated or received with a virtual reality headset or a vehicle. In both scenarios, the monitoring of a living organism can be necessary in order to ensure a secure use of virtual reality (vr) technology and secure control over a vehicle of the living organism, especially in the context of driver monitoring the security aspect is expanded to the surrounding of the living organism. To do so, no direct contact to the living organism needs to be established and the living organism, preferably human, is not limited in any movement.

From the at least two pattern features, a feature contrast is determined (120). At least one pattern feature may be comprised in one of the at least two reflection images. A feature contrast can be determined for two of the at least two pattern features by dividing the standard deviation of the intensity of one pattern feature by the mean intensity of one pattern feature, thereby determining at least two feature contrast values based on the at least two pattern features. To do so, the image may be divided into several parts. These parts may be of any shape, preferably a shape with which the full image may be covered without overlapping. In an exemplary scenario, the reflection image may be divided into squares with a size of 7 by 7 pixels. For each pixel one can then determine the feature contrast by moving the square across the reflection image and so, determining the feature contrast for every pixel. This is known as spatial laser speckle contrast imaging (LSCI). Another method for determining the feature contrast is dynamic LSCI. In dynamic LSCI the shape may be moved in the temporal axis by determining a standard deviation of intensity and the mean intensity of at least two images from different points in time. By doing so, for example the mean intensity is determined for the pixels in at least a part of a first image and the pixels in at least a part of a second image.

Since the feature contrast depends on the motion of the object, blood perfusion may be observed in one reflection image. A fast motion refers to a low contrast due to blurring of the pattern feature reflected by the moving object. A prerequisite for detecting only the motion due to blood perfusion is that the living organism from which the reflection image is generated is not moving relative to the camera. If avoiding motion not related to body fluids is not possible, the reflection images may be corrected. Determining the feature contrast as described is performed for every image. In the exemplary scenario of vr technology, a motion correction might not be necessary, since the movement of the living organism relative to the vr headset is only related to motion in the interior of the body such as blood perfusion. Regarding the movement of a living organism with control over a vehicle motion correction may be deployed to ensure that all the generated reflection images only comprise feature contrast due to moving body fluids such as blood perfusion. In some embodiments, no motion correction is needed since the driver is not moving constantly. Depending on the quantity of movements of the driver, reflection images may be generated without the driver moving because a single reflection image may be taken within several milliseconds (e.g. 1-5 ms) and thus, only a small fraction of a second is sufficient for a determination of a condition measure such as the heart rate.

In a next step, a condition measure is determined based on the feature contrast (130). By determining a regular change in feature contrast a length of a period referring to the heartbeat can be recognized. In the case where the first reflection images has been generated at point t and the second reflection image has been generated at point t+p, the associated interval is p. From this, the length of a period referring to the heartbeat may be determined. The change in blood perfusion may refer to a change in blood perfusion of at least a part of the reflection image. A part of the reflection image may refer to at least a part of the body of a living organism at different points in time. The parts of the at least two reflection images may comprise the same part of the body of a living organism. In some embodiments, at least one or more than one condition measure may be determined such as heart rate, blood pressure and/or aspiration level to provide more meaningful results based on the combination of different factors all related to the condition of a living organism. Another condition measure may be the presence of sweat, preferably the amount of sweat. When generating the reflection images of a living organism with sweat on the skin, the sweat may be more reflective than the normal skin and may not allow as much of the coherent electromagnetic radiation to penetrate the skin. Thus, more of the radiation may be reflected from not moving particles. This may result in an increased intensity of the specular reflection being the part of the pattern feature directly reflected without penetrating the skin. This way, the amount of sweat can be determined by analyzing the ratio of specular reflection and the reflection due to scattering at moving particles, such as blood.

In a last step, the condition measure is output (140). The condition measure may be output such that it can be provided to an external system, another part of the system for monitoring a living organism or an external device. In other embodiments, the condition measure may be transmitted to another part of a device via an interface. The condition measure may be transmitted to a condition control system.

In some embodiments, these steps starting with the reflection image generation may be carried out by an external device such as a cloud infrastructure. After completing at least some of the steps, the resulting feature contrast and/or condition measure may be output to a device to use it.

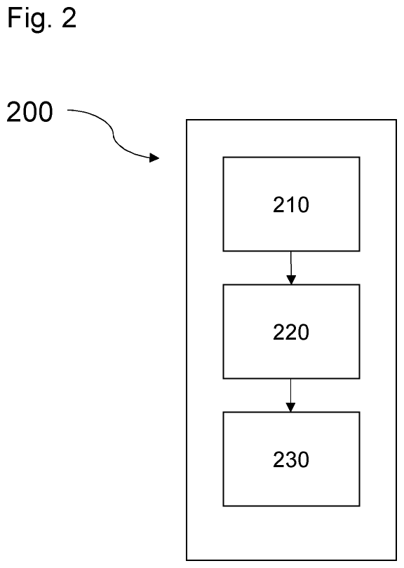

FIG. 2 illustrates a block diagram of an exemplary system for monitoring a living organism (200) comprising an input (210), a processor (220) and an output (230).

Input (210) and/or output (230) may be connected to another device or to other parts when the system is integrated into a device. Such a connection may be wired or wireless. The connection may be established via a wired or wireless connection such as one of ethernet, USB, LAN, WLAN or the like.

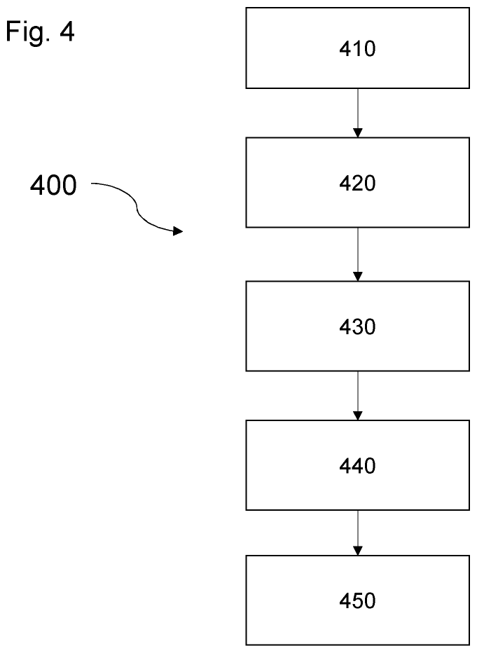

The processor (220), preferably, is configured for executing the method steps as described with reference to FIG. 1 or 4. The input (210) may be configured as an input of the processor (220) for receiving reflection images. The output (230) may be configured as an output of the processor (220). The reflection images may be generated by a camera in some embodiments.

Preferably, the processor (220) comprises a logical circuit for processing the reflection images or signals associated with the reflection images. The processor (220) is configured for determining a feature contrast of the pattern shown in the reflection image. For determining the feature contrast of the pattern, the processor (220) is configured to calculate the standard deviation of the illumination divided by the mean intensity. A resulting feature contrast value generally lies in the range between 0 and 1. A feature contrast value of 1 indicates no blurring of the pattern features, i.e., no motion in the illuminated volume of the object, and a feature contrast value of 0 indicates maximum blurring of the pattern features due to detected motion of particles, e.g., red blood cells, in the illuminated volume of the object. The processor (220) is further configured for determining a condition measure based on the determined feature contrast. The processor (220) may provide the condition measure to the providing unit (230).

To this end, the processor (220) has a neural network module comprising trained neural network. The neural network is trained for predicting the condition measure for the reflection image based on the determined feature contrast. Accordingly, the neural network is trained to use a feature contrast determined by the processor as input and to provide a condition measure as output. The trained neural network may be, for example, a multi-scale neural network or a recurrent neural network (RNN) such as a gated recurrent unit (GRU) recurrent neural network or a long short-term memory (LSTM) recurrent neural network. Alternatively, the neural network may be a convolutional neural network (CNN).

Alternatively or additionally to a trained neural network, the processor (220) may comprise an algorithm that may implement a mechanistic model. The mechanistic model is configured for determining the condition measure using based on first-principle assumptions.

In some embodiments, the system may further comprise an illumination source for illuminating an object. The object may be illuminated with light, preferably patterned IR light while a plurality of reflection images is generated.

In some embodiments, the system may further comprise a memory for storing at least one threshold.

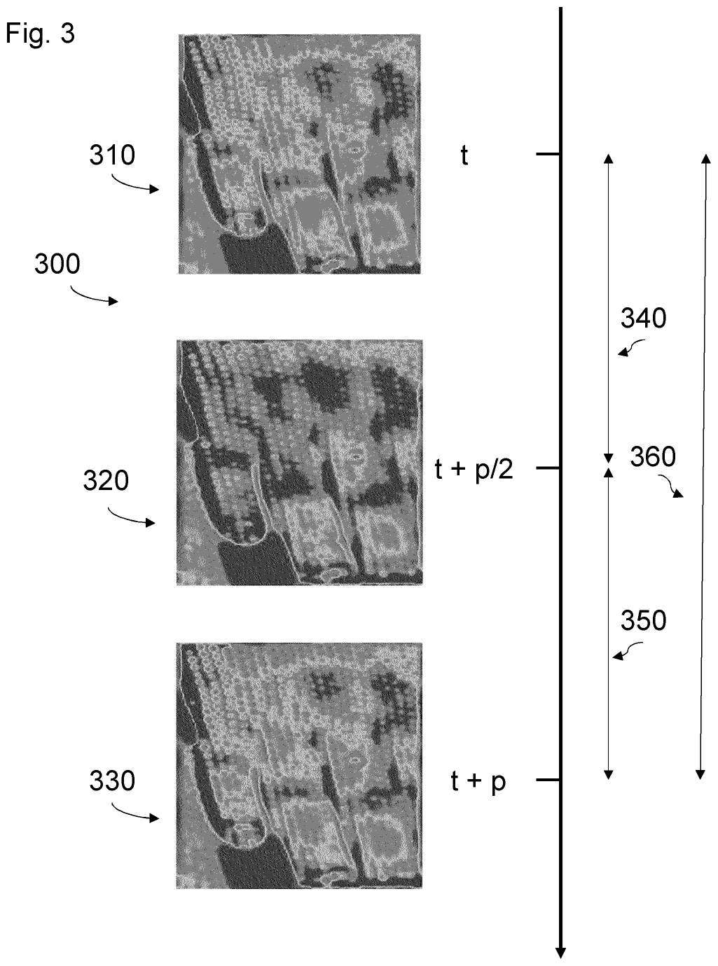

FIG. 3 shows a representation of the feature contrast as determined from reflection images at different points in time (300). In particular, a plurality of feature contrast values associated with the hand of the living organism at different points in time may be represented. This representation may be suitable for representing blood perfusion at different points in time. This representation corresponds to a contrast map. As mentioned in the context of FIG. 1, a single image can be evaluated to yield a measure of blood perfusion due to the contrast. This can be represented by a blood perfusion map (300) as shown in FIG. 3. This map (300) can be colored according to the feature contrast, wherein a higher feature contrast value is represented with dark color and a lower feature contrast value is represented with a lighter color. The spatial orientation of each pixel of the picture corresponds to the spatial orientation of a pixel of the reflection image with the corresponding feature contrast as determined.

As an example, in FIG. 3 the temporal evolution of blood perfusion in a hand (300) can be seen. By generating several reflection images at different points in time, the blood flow and the heart rate can be visualized. Part (310) of FIG. 3 is a representation of a contrast map at point in time t showing a hand with a lower blood perfusion. Due to the heart activity responsible for pumping blood into the bloodvessels, the blood perfusion increases to a maximum in the hand at point in time t+p/2 (320) where half of a period of the heart cycle passed. After reaching the maximum, the blood perfusion decreases until the initial blood perfusion is reached (330). The change in blood perfusion is visualized by the change in color wherein a high amount of light color corresponds to a high amount of blood perfusion due to low feature contrast values and a high amount of dark color corresponds to a low amount of blood perfusion due to high feature contrast values. The time passing between t and t+p/2 (340) is equal to the time between t+p/2 and t+p (350) wherein p corresponds to the length of a period. Both intervals have a length of p/2. This is represented in FIG. 3 with arrows between the points in time. Another interval providing sufficient indication of time that has passed between at least two reflection images is the interval (360) of length p between the first reflection image (310) and the last reflection image (330). By recognizing a contrast change from the minimum to the maximum blood perfusion the interval of length p/2 can be used to determine the frequency of the heart rate. Alternatively, the interval of a full cycle of length p can also be used to determine the frequency of the heart rate, also called the motion frequency.

FIG. 4 illustrates a flow diagram of an example embodiment of a method for monitoring a living organism further comprising the generation of a signal indicating a condition-based action.

In a first step, at least two reflection images are received as described in the context of FIG. 1.

In a next step, feature contrast of at least one pattern features is determined as described in the context of FIG. 1.

In a next step, a condition measure is determined based on the feature contrast as described in the context of FIG. 1.

In some embodiments, the condition measure may not be output. Instead, the condition measure may be used for a comparison with a threshold. In other embodiments, the condition measure may be output to an external system such as a cloud infrastructure. In such an infrastructure the following steps may be carried out.

In a next step, a signal indicating a condition-based action based on a comparison of the condition measure with a threshold is generated. Such a condition-based action may be used to ensure secure application or controlling of devices such as vr headsets or vehicles. In the vr example, advice to take a break or drink something may be deployed as well as ending the current application after a time limit or changing (or advice to change) the application used with the vr headset.

Similarly, in the vehicle scenario advice to take a break, drink something or change the driver or the route to a less demanding may be deployed as well as limiting the maximum velocity, turning lights or music on or off, regulating the temperature or the like.

In a last step, the signal indicating a condition-based action based on a comparison of the condition measure with a threshold is output. The signal may be an input for a condition controlling system. The signal may be transmitted back to the reflection image generator. There, the signal may be used to operate a condition control system.

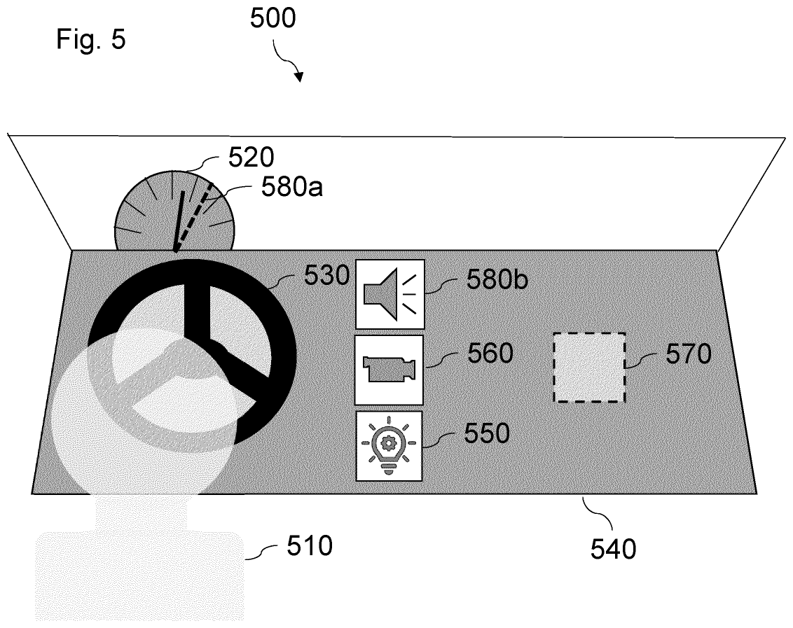

FIG. 5 illustrates an example implementation of embodiments of the invention in vehicles (500). For this purpose, the embodiments of the system may be integrated in a vehicle such as a car and suitable for carrying out the steps of the method as described above. In the concrete example illustrated in FIG. 5 a human (510), specifically the driver, may be controlling the vehicle, e.g. by stepping on the gas to achieve a velocity increase as it may be seen on the speedometer (520) or the driver may be steering with a steering wheel (530). To ensure a safe drive the vehicle can be equipped with a system for monitoring a condition of a living organism as described in the context of FIG. 2. The system for monitoring a condition of a living organism comprises an input, a processor and an output. The input and the output may be connected via a wired or wireless connection to the processor. The input may be an interface to e.g. a camera used for generating the at least two reflection images. The output may be an interface to e.g. another processor for further processing the condition measure. The condition measure may be used in a condition controlling system. In some embodiments, the condition measure may be generated in a condition controlling system. The system for monitoring a condition of a living organism may be comprised in the condition controlling system. A signal indicating a condition-based action is generated based on a comparison of the condition measure with a threshold. For this purpose, the condition controlling system comprises: an input, a processor (570) and an output. The processor (570) of the condition controlling system is suitable for generating a signal indicating a condition-based action. The processor may further be suitable for determining the condition measure based on the feature contrast. The signal indicating a condition-based action may be suitable for operating a device, a device part or a device unit such as a speaker to generate a warning signal. In some embodiments, the condition controlling system may be integrated into the dashboard (540) and may comprise an illumination source (550), a camera (560), a processor (570) and a device, device part or unit suitable for performing a condition-based action in the form of limiting the maximum velocity (580a) and/or generating a warning signal with a speaker (580b). For determining the suitability of the driver, the system may monitor a condition measure such as the heartbeat indicating a condition of the living organism. To determine the heart rate, at least two reflection images and an indication of an interval may be received. The reflection images may be generated with a camera (560) while the driver is illuminated with NIR light from an illumination source (550). In an exemplary scenario the driver may be illuminated for several seconds to generate a time series of reflection images and obtain at least one indication about interval between the different points in time where the reflection images have been generated. The time series may be generated with a specific imaging frequency of e.g. 5 frames per second. This frequency is sufficient large to fulfill the requirements of the Nyquist theorem. The driver may be illuminated with patterned coherent light such as a point cloud in the facial region or another part of bare skin which can be illuminated. To do so, an illumination source (550) may be placed in a distance between 10 to 200 cm to the driver, e.g. the illumination source (550) may be integrated in the speedometer, steering wheel or dashboard. The illuminated light may be reflected by the skin of the driver and recorded as pattern features in reflection images. For this purpose, a camera (560) may be used that can also be integrated in the speedometer, steering wheel or dashboard. The camera (560) may be equipped with image sensors sensitive to IR or NIR light. The image sensors may sense the reflected light thereby generating reflection images showing at least one pattern feature. Such reflection images may be received by an input and transmitted to a processor (570). The processor (570) may be suitable for determining a feature contrast by dividing the standard deviation σ of the mean intensity by the mean pattern feature intensity <I>. The so determined feature contrast contains information on the motion of the illuminated object. In the case of skin motion may occur due to blood perfusion. Low contrast may correspond to a fast motion. In some scenarios, the driver may be turning the head or perform other movements during the drive. Therefore, a motion correction may be used to compensate for the motion not related to blood perfusion. For this purpose, methods and devices are known in the state of the art. By determining feature contrast of the at least two reflection images the temporal evolution of blood perfusion can be followed. With the indication of at least one interval between the at least two points in time where the reflection images have been generated, a frequency for the heart rate can be determined since with the at least two reflection images and the at least one indication of the interval the frequency for a cycle of the heart pumping can be determined. Further condition measures such as blood pressure or aspiration level may be associated with information how the coherent electromagnetic radiation interacts with the skin comprised in the reflection images and the at least one interval. For better results, in the case of blood pressure, reference measurements or reference values may be used to adjust the values increasing accuracy. To determine the blood pressure and the aspiration level, a data-driven or mechanistic model may be deployed. Mechanistic models are known in the state of the art. Data-driven models may be trained according to training data sets. Such training data sets may comprise historical data sets comprising reflection images and/or feature contrast values, indication of at least one interval and condition measures corresponding to the reflection images. Such models may be available to the processor and (570) may be used to determine a condition measure. Since the condition measure is an indication of a condition of a living organism, the condition measures may be suitable for monitoring the driver. If the condition measures overcome a threshold, considered as critical value for a condition measure, the risk for an unsecure drive may be high. In an example, a stressed driver (510) may be identified via a high heart rate and/or high blood pressure. This may be especially dangerous during a drive with a large velocity of the vehicle. In an example, the threshold may be selected to be 110 as the critical value for the heart rate of a driver and the condition measure of the driver may be 120 beats per minute, a condition-based action may be triggered. In the example, the driver (510) may be driving with an increased velocity of 180 km/h. The condition controlling system may generate a signal indicating a condition-based action such as limiting the maximum velocity to 130 km/h (580a) or generating a warning signal with a speaker (580b). This disables the driver (510) to increase the velocity to values larger than 130 km/h as long as the condition measure, specifically the heart rate, overcomes the threshold selected for the heart rate of the driver (510). To do so, the vehicle may brake until the velocity is equal of smaller than 130 km/h and disable acceleration to velocities larger than 130 km/h. The threshold is selected such that the driver (510) has the chance to calm down by decreasing the stressful impact of large velocities on him.

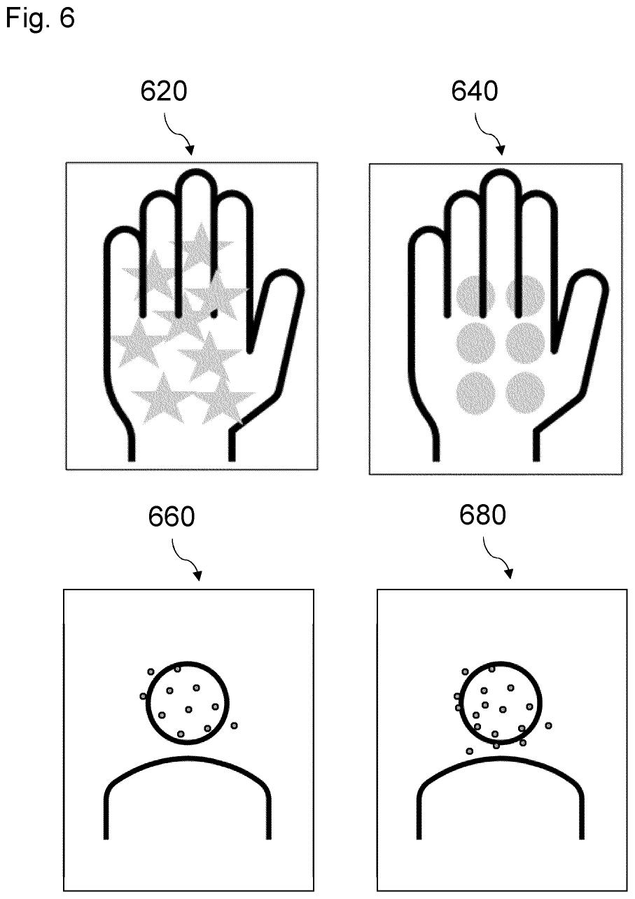

FIG. 6 illustrates an example embodiment of a reflection image in relation to the contours of the living organism (620, 640, 660, 680). A reflection image shows at least a part of a living organism under illumination by patterned coherent electromagnetic radiation. For this purpose, the living organism may be illuminated with patterned coherent electromagnetic radiation. Patterned coherent electromagnetic radiation may comprise at least one light beam. The at least one light beam may be projected onto the living organism. The projection of the at least one light beam onto the living organism may result in illuminating at least one contiguous area with coherent electromagnetic radiation. A reflection image may be generated from the light reflected from the living organism illuminated by the at least one light beam. The at least one contiguous area of coherent electromagnetic radiation may be seen in the reflection image, eg in the form of a light spot. The light spot in the reflection image may be referred to as a pattern feature. In FIG. 6 four examples for a reflection image in relation to the contours of the living organism are shown (620, 640, 660, 680). 620 may refer to a reflection image generated while a hand of the living organism is illuminated by patterned coherent electromagnetic radiation. In the example 620, the pattern features may be star-shaped and may be arranged irregularly. The hand in 620 may be sketched to outline that the reflection image may be generated while the hand of the living organism was illuminated by patterned electromagnetic radiation. The reflection image may be independent of the contours of the hand of the living organism. Similarly, 640 shows six circular light spots that have been reflected by the hand of the living organism. Followingly, the pattern associated with the patterned electromagnetic radiation may be a regular dot pattern with rectangular symmetry. In 660 the face of the living organism was illuminated with patterned coherent electromagnetic radiation. The projection of the patterned coherent electromagnetic radiation may result in a regular arrangement of pattern features, eg in a hexagonal arrangement. The pattern associated with the patterned coherent electromagnetic radiation may be a hexagonal pattern. In 680 the face of the living organism was illuminated with patterned coherent electromagnetic radiation. The projection of the patterned coherent electromagnetic radiation may result in an irregular arrangement of pattern features. This may be achieved by randomly arranging light beam emitters in an illumination source and/or using different optical elements in relation to light beams associated with the patterned coherent electromagnetic radiation.

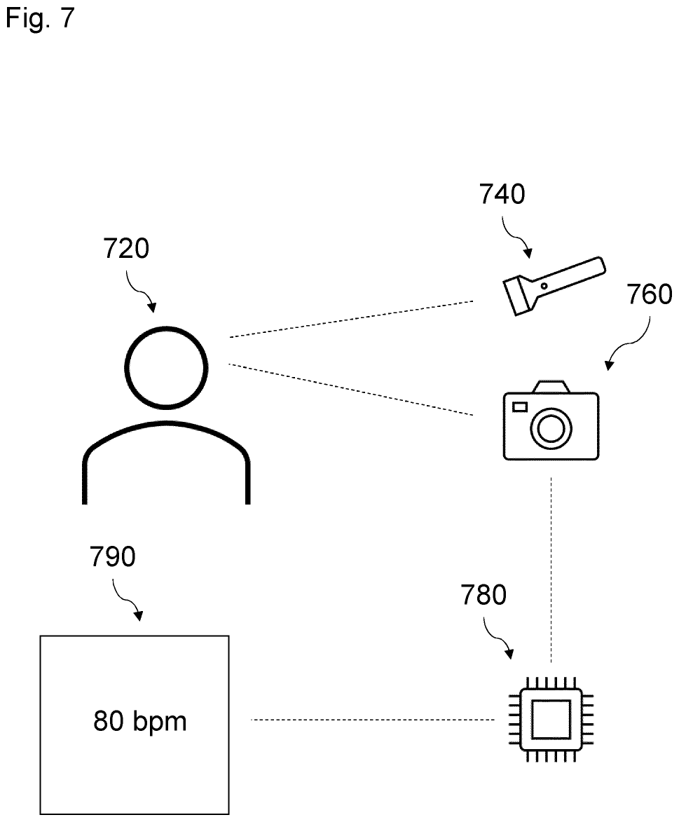

FIG. 7 illustrates an example application of the methods and systems as described herein. The system as described in the context of FIG. 2 and the method as described in the context of FIG. 1 may be used for patient monitoring. In such an embodiment, a patient 720 may be illuminated by patterned coherent electromagnetic radiation emitted from an illumination source 740. Preferably, the patient may be at rest, eg placed in a bed. The illumination source may comprise a VCSEL array and a DOE for duplicating the light beams emitted by the VCSEL array. Hence, the illumination source may emit a plurality of coherent light beams. The plurality of coherent light beams—also referred to as patterned coherent electromagnetic radiation may be projected onto the patient 720. Followingly, the patient 720 may be illuminated with a light pattern. The light may interact with the skin of the patient 720. The interaction between the coherent light and the skin of the living organism such as the patient 720 may result in the formation of speckles. Two reflection images showing the patient under illumination with coherent electromagnetic radiation may be generated with a camera 760 at two different points in time. The reflection images may show the projection of the plurality of light beams onto the skin of the patient 720 resulting in a plurality of pattern features. Preferably, the number of pattern features may be equal to the number of light beams associated with the patterned coherent electromagnetic radiation. The reflection images may be associated with a timestamp. From the timestamps associated with the reflection images, an indication of the at least one interval between the at least two different points in time may be derived. The indication of the interval and the reflection images may be provided to the processor 780 through an input. Processor and input may refer to the processor and input as described in the context of FIG. 2. The processor may perform the method as described in the context of FIG. 1. The condition measure may be outputted as described in the context of FIG. 1, eg via an output of a system as described within the context of FIG. 2. In an example, the condition measure may be provided to a display 790. The display 790 may be suitable for displaying the condition measure, in particular the values and/or units associated with the condition measure. The display 790 may be used for visual tracking of the condition of the patient 720.

As used herein “determining” also includes “initiating or causing to determine”, “generating” also includes “initiating or causing to generate” and “providing” also includes “initiating or causing to determine, generate, select, send or receive”. “Initiating or causing to perform an action” includes any processing signal that triggers a computing device to perform the respective action. Any disclosure and embodiments described herein relate to the methods, the systems, devices, the computer program element lined out above and vice versa. Advantageously, the benefits provided by any of the embodiments and examples equally apply to all other embodiments and examples and vice versa.

In the claims as well as in the description the word “comprising” does not exclude other elements or steps. The indefinite article “a” or “an” and the definite article “the” does not exclude a plurality. In particular, indefinite article “a” or “an” may be replaced with one or more and the definite article “the” may be replaced with the one or more. A single element or other unit may fulfill the functions of several entities or items recited in the claims. The mere fact that certain measures are recited in the mutual different dependent claims does not indicate that a combination of these measures cannot be used in an advantageous implementation.

1. A method for monitoring a condition of a living organism comprising:
(a) receiving an image set comprising at least two reflection images generated at different points in time and an indication of at least one interval between at least two different points in time, wherein a reflection image is generated while the living organism is illuminated by patterned coherent electromagnetic radiation, and, wherein the reflection image shows at least one pattern feature formed by illuminating at least a part of the living organism by the patterned coherent electromagnetic radiation, and;
(b) determining a feature contrast of the at least two pattern features;
(c) determining a condition measure of the living organism based on the feature contrast and the indication of the at least one interval; and
(d) outputting the condition measure.

2. The method according to claim 1, wherein the image set comprises at least three reflection images and wherein the at least three reflection images have been generated in a time series with a constant time interval associated with an imaging frequency or changing time intervals associated with a mean imaging frequency.

3. The method according to claim 2, wherein the imaging frequency is at least twice the motion frequency being associated with an expected periodic motion of a body fluid.

4. The method according to claim 1, wherein a condition measure of the living organism based on the feature contrast and the indication of the at least one interval is determined by providing the feature contrast of the at least two pattern features and the indication of the at least one interval between the generation of the at least two pattern images to a data-driven model, wherein the data-driven model is parametrized on a training data set including historical feature contrasts, historical indications of the at least one interval and historical condition measures.

5. The method according to claim 1, wherein the reflection images are motion corrected based on motion tracking data associated with the movement of the living organism.

6. The method according to claim 1, wherein the coherent electromagnetic radiation has a wavelength between 900 nm and 1000 nm.

7. The method according to claim 1, wherein the patterned coherent electromagnetic radiation are emitted from an illumination source and wherein the image set comprising at least two reflection images is received from a camera, and, wherein a distance between the living organism and the camera and/or the distance between the living organism and the illumination source is 5 cm and 150 cm.

8. The method according to claim 1, wherein the condition measure comprises at least one of the heart rate, the blood pressure and/or the aspiration level.

9. The method according to claim 1, wherein outputting the condition measure is substituted by:
generating a signal indicating a condition-based action based on a comparison of the condition measure with a threshold associated with a critical condition measure, and
outputting the signal indicating the condition-based action.

10. The method according to claim 9, wherein the threshold is specific for the living organism.

11. A non-transitory computer-readable data medium storing a computer program including instructions for executing steps of the method according to claim 1.

12. A system for monitoring a condition of a living organism comprising:
(a) an input for receiving an image set comprising at least two reflection images generated at different points in time and an indication of at least one interval between at least two different points in time, wherein a reflection is generated while the living organism is illuminated by patterned coherent electromagnetic radiation, and, wherein the reflection image shows at least one pattern feature formed by illuminating at least a part of the living organism by the patterned coherent electromagnetic radiation;
(b) a processor for
determining a feature contrast of the at least two pattern features; and
determining a condition measure of the living organism based on the feature contrast and the indication of the at least one interval; and
(c) an output for outputting the condition measure.

13. The system according to claim 12, wherein the system further comprises a camera for generating the at least two reflection images and/or an illumination source for illuminating the living organism by the patterned coherent electromagnetic radiation.

14. A method of using the condition measure obtained by the method according to claim 1, the method comprising using the condition measure in a condition controlling system.

15. A condition controlling system comprising:
(a) an input for receiving a condition measure obtained by the method according to claim 1 and a threshold associated with a critical condition measure;
(b) a processor for generating a signal indicating a condition-based action based on a comparison of the condition measure with the threshold associated with a critical condition measure; and
(c) an output for outputting the signal indicating a condition-based action.
